(19) United States
(12) Patent Application Publication
(10) Pub. No.: US 2025/0035605 A1
(43) Pub. Date: Jan. 30, 2025

(54) METHOD TO DETERMINE THE PACKAGING TIME OF AN EDIBLE OIL EXTRACTED FROM OLIVES

(71) Applicant: AGRITALIA S.P.A., Napoli (IT)

(72) Inventor: Sergio MASSA, Sant'Agnello (NA) (IT)

(73) Assignee: AGRITALIA S.P.A., Napoli (IT)

(21) Appl. No.: 18/709,044

(22) PCT Filed: Jun. 8, 2023
(86) PCT No.: PCT/IB2023/055945

§ 371 Date: May 10, 2024

(30) Foreign Application Priority Data
Jun. 9, 2022 (IT) 102022000012257

(51) Int. Cl. G01N 33/03; G01N 1/22; G01N 30/02; G06N 20/20

(52) U.S. Cl. CPC G01N 33/03; G01N 1/2226; G01N 30/02; G06N 20/20; G01N 2001/2229; G01N 2030/025

(57) ABSTRACT

A method, implemented by at least one computer, for determining the time which has elapsed since the packaging of a packaged edible oil, which is extracted from olives, includes:
training at least one machine learning model to determine the time that has elapsed since the packaging of a packaged edible oil extracted from olives,
analyzing a sample of the oil, to acquire the quantity/concentration data of a plurality of analytes contained in the oil sample, the oil sample being analyzed by a headspace technique coupled with a gas chromatograph and spectrometer,
sending, the data acquired in this way, and relating to quantities/concentrations of the analytes in the sample of oil, at an input to the at least one machine learning model, already trained, to thus obtain, at an output, a numerical value corresponding to, or representative of, the time which has elapsed since the packaging of the oil.

### CROSS REFERENCE TO RELATED APPLICATIONS

This application is a § 371 U.S. National Phase of International Patent Application No. PCT/IB2023/055945, filed Jun. 8, 2023, which claims priority of Italian Patent Application No. 102022000012257, filed Jun. 9, 2022, the entire contents of all of which are incorporated by reference herein as if fully set forth.

### FILED OF THE INVENTION

The present invention relates to a method, implemented by means of at least one electronic processor, such as for example a processor/controller/computer, for determining the time which has elapsed since the packaging of a packaged edible oil extracted from olives, preferably of a packaged virgin or extra-virgin oil.

The present invention also relates to a method, implemented by means of at least one electronic processor, such as for example a processor/controller/computer, for training a machine learning model in order to determine the time which has elapsed since the packaging of a packaged edible oil extracted from olives, preferably of a packaged virgin or extra-virgin oil.

### BACKGROUND

As is known, virgin and extra virgin olive oil, which are its two possible product forms suitable for sale, is a lipid made up of over 80% triglycerides of unsaturated fatty acids and has a natural tendency to oxidize, thus losing its freshness over time. In particular, as it ages, virgin and extra virgin olive oil completely loses its edibility characteristics, as established by Regulation 2568/1991 and its subsequent amendments, thus returning to the class of lampante, which is not suitable for consumption.

The producers, processors and packers of virgin and extra virgin olive oil are committed to finding solutions to slow down the oxidative process of the oil over time as much as possible. However, the oxidative chain inevitably always produces a free radical which attacks the unsaturated bond of the still intact fatty acids and a series of molecules derived with a submultiple number of carbon atoms of the starting fatty acids (from 18C to 6C) belonging to the family of alcohols, aldehydes and ketones.

The shelf-life of an extra virgin olive oil—understood as the period of time in which the oil itself has a quality compliant with the legal limits—is naturally established by a series of factors including: origin, cultivar, composition in antioxidants (whether they are phenols or tocopherols), methods of extraction, conservation and distribution.

Usually, in the distribution reality, the shelf-life of an oil is super-imposed by the distributor according to his logistical needs and distribution rotation. Faced with this significant constraint imposed by the distributor, it is then up to the packer to “create” ad hoc oils to meet the shelf-life needs of the distributor, and in particular this is obtained by mixing together different types of oils, i.e. obtained from different variety of cultivated olives (cultivars), in order to optimize the shelf-life, as well as other aspects such as production costs and sensory profile.

In this context there is therefore the need to determine, or rather predict, the shelf-life of a virgin or extra virgin olive oil, in particular before placing it on the market.

At an academic level, various studies on oxidation phenomena in virgin olive oils have already been proposed, thus leading to the development of shelf-life models or accelerated shelf-life models according to the Arrhenius equation (linear regression).

In known shelf-life models, the critical parameters considered have generally been the following: free fatty acids (FFA), peroxide index (PV), ultraviolet absorption (K232, K270), evaluation of diglycerides (DAG's), evaluation of pyrophaeophytins (PPP), sensory profile, induction time (Rancimat Metrohm), total phenols and fatty acid profile (FAP).

In general, the well-known predictive models of the olive oil shelf-life are based on the observation of the variations of a single chemical-physical parameter under the conditions of the natural or accelerated shelf-life of the olive oil, also selecting the limits of this parameter which are acceptable for shelf-life purposes.

Furthermore, over the years various predictive models of the shelf-life of olive oil have already been proposed, most of which are based on analytes attributable to the oil. For example, the Oleum 2020 project of the European Union envisaged the development of a software, called “VITA OLEI” @ (University of Perugia), to predict the minimum conservation term (also called “TMC” or “Best before”) based on the following input parameters: sample name, storage condition (light/dark and temperature) and a selection of analytical parameters. The software output is represented by the time (expressed in days) beyond which the threshold value is exceeded. The accuracy of the output forecast thus provided is suboptimal, and is at best satisfactory to provide a tool for producers' needs and to strengthen consumer confidence.

Another well-known study, which resulted from the collaboration between the University of Udine and INNOV-HUB (formerly the Oil and Grassi Experimental Station of Milan), verified the application of the accelerated shelf-life method (ASLT).

In general, the well-known published studies are based on linear models (for example “Ordinary Least Square” and/or “Partial Least Square”) which are generally applied to input data of the targeted type, i.e. precisely determined and selected, and generally they relate only to the phenolic, tocopherol, acid and triglyceride fractions.

### OBJECTS OF THE INVENTION

The object of the present invention is to propose a method, implemented by means of at least one electronic processing unit, such as for example a processor/controller/computer, which allows the drawbacks of traditional solutions to be overcome, at least in part.

Another object of the present invention is to propose a method, implemented by means of at least one electronic processor, such as for example a processor/controller/computer, which allows to determine the time which has elapsed since the packaging of a packaged edible oil extracted from olives, preferably a packaged virgin or extra-virgin oil.

Another object of the present invention is to propose a method, implemented by means of at least one electronic processor, such as for example a processor/controller/computer, which allows to determine in a simple, accurate and precise way the time which has elapsed since the packaging of an edible oil packaged extracted from olives, preferably of a packaged virgin or extra-virgin oil.

Another object of the present invention is to propose a method which allows to determine in a simple and repeatable way the time which has elapsed since the packaging of a packaged edible oil extracted from olives, preferably of a packaged virgin or extra-virgin oil.

Another object of the present invention is to propose a method which allows to determine the minimum conservation term (TMC) of a packaged edible oil extracted from olives, preferably of a virgin or extra-virgin oil.

Another object of the present invention is to propose a method which is highly robust and reliable.

Another object of the invention is to propose a method which can be implemented simply, rapidly and at low cost.

Another object of the invention is to propose a method which is an improvement and/or alternative to any other traditional solutions.

Another object of the invention is to propose a method which has an alternative characterization with respect to the traditional solutions.

Another object of the invention is to propose a method which can be implemented and used in devices having hardware of the traditional type and which is already currently available.

### SUMMARY OF THE INVENTION

All these objects, both alone and in any combination thereof, and others which will result from the following description are achieved, according to the invention, with a method as defined in the appended claims.

### BRIEF DESCRIPTION OF THE FIGURES

The present invention is hereinafter further clarified in some of its preferred embodiments shown for purely exemplifying and non-limiting purposes with reference to the attached tables of drawings, in which:

FIG. 1 shows a flowchart of the method according to the invention during the training phase,

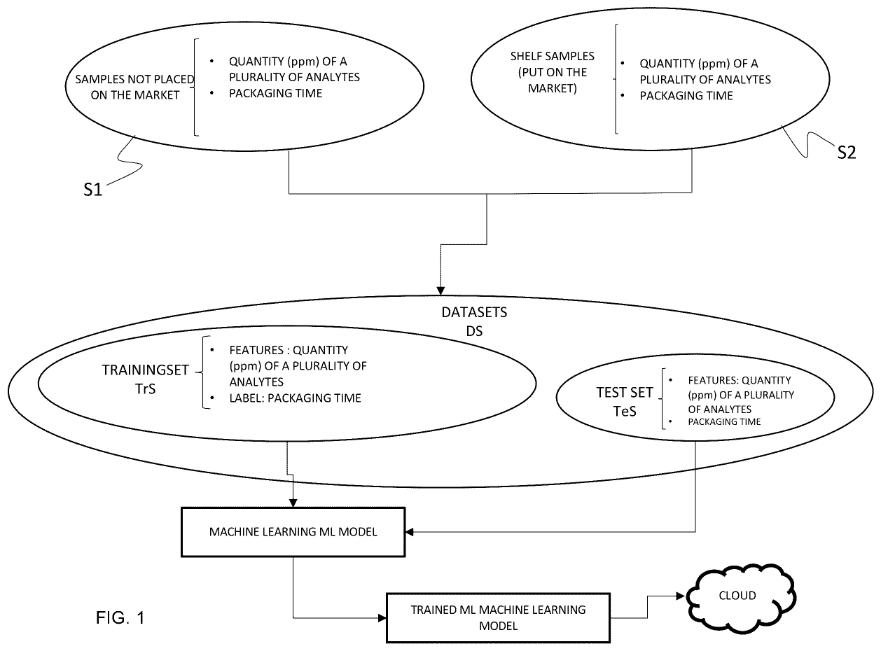

FIG. 2 shows a flowchart of the method according to the invention during the inference/prediction step, and

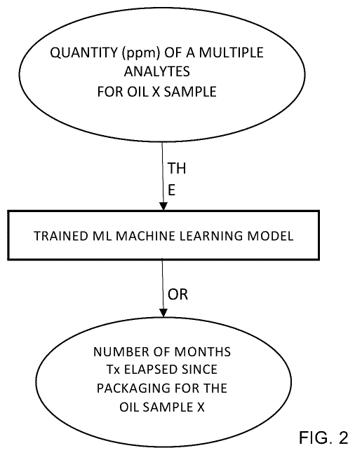

FIG. 3 shows an example of the data format of the dataset DS, and preferably of the training set TrS, in the method according to the invention.

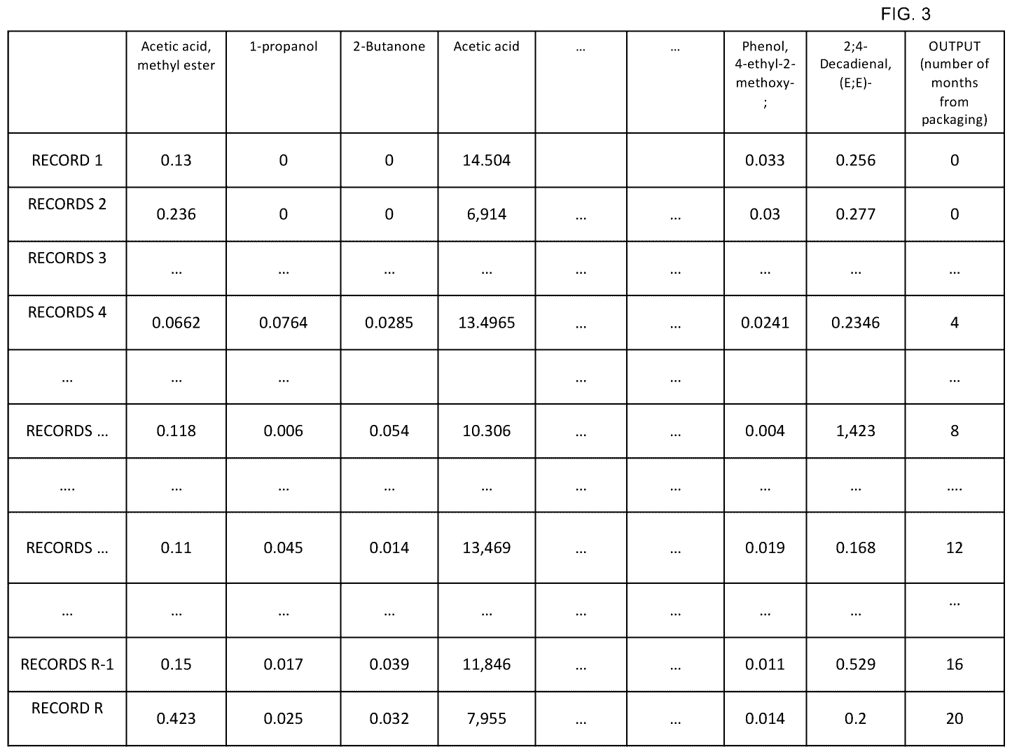

### DETAILED DESCRIPTION OF THE PREFERRED EMBODIMENTS

The present invention relates to a method, implemented by at least one processor, such as for example a processor, a controller or a computer, for determining the time which has elapsed since the packaging of an edible oil which is packaged and which is extracted from olives, preferably a virgin and/or extra-virgin oil.

Conveniently, “virgin oil” and “extra-virgin oil” mean an edible oil having the characteristics set forth in the relevant regulations.

Preferably, the packaged edible oil is extracted from olives exclusively mechanically, or predominantly mechanically.

Conveniently, the packaged edible oil can be monovarietal (monocultivar), and thus be obtained from a single variety of cultivated olive (cultivar), or it can be obtained from the mixing/union (blend) of different oils, obtained from different varieties of cultivated olives (cultivars).

Preferably, the packaging is carried out by bottling. Conveniently, it is understood that the packaging can be carried out by placing the oil inside any container, for example metal, for storage and sale.

### INFERENCE

In particular, according to the invention, the method comprises an inference step in which at least one ML machine learning model is executed which has been previously trained in order to determine the time which has elapsed since the packaging of a packaged edible oil extracted from olives, preferably of a virgin or extra-virgin oil.

Preferably, the training of said at least one ML machine learning model is performed off-line. Preferably, once trained, said at least one ML machine learning model can be uploaded to the cloud or to a shared remote unit accessible from at least one local terminal, for example via the web.

The present invention relates to a method, implemented by at least one processor, such as for example a processor, a controller or a computer, for determining the time which has elapsed since the packaging of an edible oil X which is packaged and which is extracted from olives, preferably of a virgin or extra-virgin oil, said method being characterized by the fact that:
at least one ML machine learning model is trained to determine the time that has passed since the packaging of a packaged edible oil extracted from olives, preferably of a virgin or extra-virgin oil,
an oil sample X is analyzed, for which it is desired to determine the time elapsed since its packaging, to acquire the quantity/concentration data of a plurality of analytes contained in said oil sample X, said oil sample X being analyzed by means of headspace technique coupled with a gas chromatograph and a spectrometer,
   the data acquired in this way, and relating to the quantities/concentrations of said plurality of analytes in the oil sample X, are sent as input I to said at least one machine learning model ML to thus obtain as output O a numerical value Tx corresponding to, or representative of the time which has elapsed since the packaging of said sample of oil X.

Preferably, in one possible embodiment thereof, the method according to the invention is characterized in that:
a first ML1 machine learning model is trained, preferably of the adaptive amplification regression type,
train a second ML2 machine learning model with an algorithm that is different than the first machine learning model, preferably train a second ML2 machine learning model with a random forest regression algorithm,
the data thus acquired, and relating to the quantities/concentrations of said plurality of analytes in the oil sample X, are sent at input I to said first machine learning model ML1 to thus obtain at output O1 a first numerical value Tx1 corresponding to, or representative of the time which has elapsed since the packaging of said oil sample X,
the data thus acquired, and relating to the quantities/concentrations of said plurality of analytes in the oil sample X, are sent at input I to said second machine learning model ML2 to thus obtain at output O2 a second numerical value Tx2 corresponding to, or representative of the time which has elapsed since the packaging of said oil sample X,
if the first numerical value Tx1 is different from the second numerical value Tx2, one of said first numerical value Tx1 and said second numerical value Tx2 is selected, and in particular one of said two values is selected on the basis of the performance indexes of the respective models, preferably based on the coefficient of determination and/or mean absolute error calculated for the respective ML1 and ML2 machine learning models.

Preferably, for each of the machine learning models ML1 and ML2 the coefficient of determination and the mean absolute error are calculated and Tx1 or Tx2 is selected depending on which of the two models ML1 or ML2—which output Tx1 and Tx2 respectively—has a higher coefficient of determination and a lower average absolute error.

Conveniently, if instead the first numerical value Tx1 is equal to Tx2, then Tx corresponds to Tx1 and Tx2, ie Tx=Tx1=Tx2.

Preferably, the calculation of Tx1 (with the corresponding performance indexes) and Tx2 (with the corresponding performance indexes) is done in sequence, i.e. first Tx1 is calculated and then Tx2 is calculated, or vice versa. Conveniently, the calculation of Tx1 and Tx2 in sequence can be performed using the same computer in which both the first ML1 machine learning model and the second ML2 machine learning model are loaded, trained and/or executed.

Preferably, the calculation of Tx1 (with the corresponding performance indexes) and Tx2 (with the corresponding performance indexes) can be done simultaneously by means of corresponding computers operating in parallel, and in which a computer is loaded, trained and/or executed first ML1 machine learning model while the second ML2 machine learning model is loaded, trained, and/or run on the other computer.

Preferably, the data of the quantities/concentrations of the individual analytes are expressed in mg/kg (ppm).

Preferably, said plurality of analytes of which—for each—the corresponding quantity/concentration is acquired are defined by a plurality of odorous analytes present in the packaged edible oil extracted from olives, preferably of a virgin or extra-virgin oil.

Preferably, said plurality of analytes of which—for each—the corresponding quantity/concentration is acquired are analyzed by means of the headspace technique with solid phase micro-extraction (SPME).

Preferably, said plurality of analytes of which—for each—the corresponding quantity/concentration is acquired are analyzed by means of the headspace technique (HS) with solid phase micro-extraction (SPME) coupled with a gas chromatograph (GC) and a mass spectrometer (MS); said technique being also known by the acronym “HS-SPME-GC/MS”.

Preferably, said plurality of analytes of which—for each—the corresponding quantity/concentration is acquired are analyzed by means of the HS-SPME-GC/MS technique with the same instrumental and experimental conditions described in “Multiple internal standard normalization for improving HS-SPME-GC-MS quantitation in virgin olive oil volatile organic compounds (VOO-VOCs) profile” by Martina Fortinia, Marzia Migliorini, Chiara Cherubini, Lorenzo Cecchi, Luca Calamai, Talanta Volume 165, 1 Apr. 2017, Pages 641-652.

Conveniently, the quantities/concentrations of said plurality of analytes present in a sample of oil X are obtained by the following procedure:
a determined quantity of the oil sample X to be analyzed is inserted into a container, for example a phial (vial) with a suitable septum/closing cap, and preferably a predefined and suitable quantity of a reference standard can also be added to the inside of said container,
an SPME fiber is exposed—preferably after having thermostated the vessel containing the oil sample to be analyzed—in the headspace of the closed vessel, for a predefined time (e.g. 20 minutes) and under stirring (e.g. at a stirring speed of 400-500 rpm),
the SPME fiber is desorbed for a predefined period (for example 2 sec—2 minutes) in the injection port of a gas chromatograph which, preferably, operates in “splitless” mode at an injection temperature of 260° C.,
the quantities/concentrations of the analytes are identified and determined, in the traditional way, by means of gas chromatography coupled to mass spectrometry.

Preferably, the SPME fiber is DVB/CAR/PDMS (Divinylbenzene/Carboxen/Polydimethylsiloxane).

Preferably, the gas chromatograph column has a thickness of 0.25 mm, a length of 56 m and a diameter of 0.25 mm.

Preferably, the gas chromatograph is configured to operate in the following modes:
oven column temperature: 35° C.;
injection temperature: 260° C.;
injection mode: splitless;
sampling time: 0.05 min;
carrier gas: He;
split ratio: 50.

Preferably, the initial temperature of the GC column is maintained at 36° C. for 15 minutes, then increased to 160° C. with an increase of 4°/minute and a holding time of 1 minute, then increased to 300° C. with an increase of 50°/minute and a holding time of about 1 minute.

Preferably, mass spectrometry is performed using a quadrupole mass analyzer.

Preferably, in another possible embodiment, the odorous analytes are analyzed by the headspace technique (HS) coupled with a gas chromatograph (GC) and an ion mobility spectrometer (IMS), also known as “HS-GC-IMS”.

Suitably, therefore, the spectrometer used for the analysis coupled with the gas chromatograph can be a mass spectrometer (MS) or an ion mobility spectrometer (IMS).

Preferably, said plurality of analytes of which—for each—the corresponding amount/concentration is acquired comprise at least ten analytes, more preferably comprise at least 50 analytes and, even more preferably, comprise 70-80 analytes.

Preferably, in a possible embodiment, said plurality of analytes of which—for each—the corresponding quantity/concentration is acquired comprise: Acetic acid, methyl ester; 1-propanol; 2-Butanone; Acetic acid; 2-butanol; Ethyl acetate; 1-propanol, 2-methyl-; methyl propionate; Butanal, 3-methyl-; Butanal, 2-methyl-; 1-penten-3-ol; 1-penten-3-one (ethyl vinyl ketone); Propanoic acid; 3-Pentanone; Pentanal; heptane; (R)-(−)-2-pentanol; Propanoic acid, ethyl ester; 1-butanol, 3-methyl-; 1-butanol, 2-methyl-; 2-penalty, (E)-; 1-pentanol; 2-penten-1-ol, (E)-; 2-penten-1-ol, (Z)—; butanoic acid; Octane; exhale; Butanoic acid; ethyl ester; Acetic acid; butyl ester; 3-hexenal, (Z)—; 2-hexenal, (E)-; 3-hexen-1-ol, (E)-; 3-hexen-1-ol, (Z)—; 2-hexen-1-ol, (E)-; 2-hexen-1-ol, (Z)—; 1-hexanol; pentanoic acid; 2-heptanone; heptanal; 2-heptanol; 2;4-hexadienal, (E;E)-; 2-Heptenal, (E)-; Benzaldehyde; 1-heptanol; 1-Oct-3-one; Hexanoic acid; Phenol; 5-heptene-2-one, 6-methyl-; 1-octene-3-ol; 2-octanone; 2-octanol; octal; 3-hexen-1-ol; acetate; (Z)—; 2;4-heptadienal, (E;E)-; Acetic acid; thin ester; 2-hexen-1-ol; acetate, (E)-; D-Limonene; 2-octal, (E)-; 1-octanol; Phenol, 2-methoxy-(Guaiacol); 2-Nonanone; Not anal; Phenylethyl alcohol; 2-Nonenal; (AND)-; Phenol; 4-ethyl-; 1-Nonanol; Decalogue; 2;4-Nonadienal, (E;E)-; 2-Decenal, (E)-; Phenol, 4-ethyl-2-methoxy-; 2;4-Decadienal, (E;E)-.

Preferably, in another possible embodiment, said plurality of analytes of which—for each—the corresponding quantity/concentration is acquired also comprise, in addition to those reported above, the following further analytes: acetic acid-d3; trimethylacetaldehyde; Ethyl acetate-d8; butanol-d10; toluene-D8; 2-pentanol, 4-methyl; 3-octanone; 6-chloro-2-hexanone; Phenol, 3,4-dimethyl.

Preferably, said at least one ML machine learning model can comprise said first ML1 machine learning model and said second ML2 machine learning model. Therefore, appropriately, what is specified below for model ML may also apply to the first model ML1 and to the second model ML2.

Preferably, the 0 output may comprise the 01 output of model ML1 and the 02 output of model ML2. Therefore, conveniently, what is specified below for the output O can also be valid for the first output O1 and for the second output O2.

Preferably, the numeric value Tx can comprise a first numeric value Tx1 and a second numeric value Tx2. Therefore, conveniently, what is specified below for the numerical value Tx can also be valid for the first numerical value Tx1 and for the second numerical value Tx2.

In particular, the output O of the ML machine learning model is representative of the time interval that has elapsed between the packaging of the oil sample X and said phase in which said oil sample X is analyzed.

Conveniently, as output O from said machine learning model ML a numerical value representative of the time Tx which elapsed between the packaging of the oil sample X and the analysis of the sample itself is obtained.

Preferably, the numerical value Tx indicates the number of months elapsed between the month in which the analysis was carried out and the month in which the packaging was carried out.

Conveniently, the numerical value Tx can be expressed in number of months, or in number of periods comprising two or more months (for example bimesters, quarters, quarters, etc.), or in number of days.

Preferably, the outgoing Tx numerical value 0 may be approximated to the integer unit.

Preferably, the numerical value Tx output O from said machine learning model ML can be further processed in order to determine the residual/missing time Tm with respect to a predefined maximum duration time Tmax (for example 18 months), where in particular Tm=Tmax−Tx.

Preferably, the numerical value Tx output O can represent and/or be used to calculate and/or evaluate the “minimum term of conservation” (TMC) which—according to the relevant regulations (for example the EU Reg. 1169/2011) must be reported on the label of food products and which is defined as “the date until which a product retains its specific properties under suitable storage conditions”. In particular, as far as extra virgin olive oil is concerned, these specific properties can be found in the legislation which defines the qualitative characteristics of an extra virgin olive oil, and more in detail in the following current regulations: EEC Regulation 2568/91 and Delegated Regulation (EU) 2015/1830).

Conveniently, the minimum conservation term (TMC), as defined in the sector regulations, can correspond to or be calculated starting from the numerical value that is provided in output OR by the ML machine learning model.

### TRAINING

The present invention also relates to a method, implemented by means of at least one processor, such as for example a processor, a controller or a computer, for training at least one ML machine learning model, preferably of the adaptive amplification regression type (also called “adaboost regression” or “adaptive boosting regression”) or of the random forest regression type (also called “Random Forest Regression”), to determine the time that has elapsed since the packaging of a sample of packaged edible oil extracted from olives, preferably of a virgin or extra-virgin.

The present invention also relates to a method, implemented by at least one processor, such as for example a processor, a controller or a computer, for training:
a first ML1 machine learning model, preferably of the adaptive amplification regression type (also called “adaboost regression”), to determine the time that has elapsed since the packaging of a sample of packaged edible oil extracted from olives, preferably of a virgin or extra virgin olive oil—virgin, and
a second ML2 machine learning model of the random forest regression type (also called “Random Forest Regression”), to determine the time that has elapsed since the packaging of a sample of packaged edible oil extracted from olives, preferably of a virgin or extra-virgin.

Preferably, as mentioned, in the inference phase, both machine learning models ML1 and ML2 are used jointly, to then compare and select only one of the numerical values Tx1 and Tx2 supplied in output by said two models, and this in order to provide output a numerical value (corresponding to, or representative of, the time which has elapsed since the packaging of said oil sample X) which is more secure and reliable.

Preferably, said at least one ML machine learning model can comprise said first ML1 machine learning model and said second ML2 machine learning model. Therefore, appropriately, what is specified below for model ML may also apply to the first model ML1 and to the second model ML2.

Preferably, the O output may comprise the O1 output of model ML1 and the O2 output of model ML2. Therefore, conveniently, what is specified below for the output O can also be valid for the first output O1 and for the second output O2.

Preferably, the numeric value Tx can comprise a first numeric value Tx1 and a second numeric value Tx2. Therefore, conveniently, what is specified below for the numerical value Tx can also be valid for the first numerical value Tx1 and for the second numerical value Tx2.

In particular, according to the invention, the method comprises a training phase of at least one ML machine learning model in order to determine the time which has elapsed since the packaging of a sample of packaged edible oil extracted from olives, preferably of a virgin oil or extra virgin.

Conveniently, said at least ML machine learning model is trained using a supervised learning DS dataset comprising quantity/concentration data of a plurality of analytes related to various oil samples which are analyzed by the headspace technique coupled with a gas chromatograph and a spectrometer at a known moment which is defined starting from the packaging date of each oil sample.

Conveniently, the various oil samples analyzed for training the ML machine learning model can be of the same type and/or of different types.

Preferably, the various oil samples analyzed for training the ML machine learning model are obtained by analyzing an oil of the same type in a plurality of successive times/moments, which are known and defined starting from the packaging date of each oil sample.

Preferably, said ML machine learning model is trained with a DS dataset for supervised learning comprising an organized collection of records (R), one for each oil sample analyzed. Each record comprises a plurality of labeled analytes, i.e. in which the quantity/concentration values of said plurality of analytes is associated with a numerical value representative of the time elapsed between the packaging date and the date on which the analysis was carried out. led to determine the corresponding values of quantity/concentration of the analytes.

Preferably, the DS dataset comprises a plurality of records (R), one for each analysis carried out on an oil sample, and each record comprises:
as characteristics (features) the quantity/concentration data relating to each analyte of said plurality of analytes analyzed by means of the headspace coupled with a gas chromatograph and a spectrometer, preferably by means of the HS-SPME-GC/MS technique,
a numerical value (label), preferably integer and expressed in months and/or days, which indicates the time elapsed between the packaging date and the date/moment in which the analysis was carried out which led to the determination of the corresponding amount/concentration of analytes.

Preferably, the data of the quantities/concentrations of the individual analytes of the DS dataset are expressed in mg/kg (ppm).

Preferably, each feature of the DS dataset corresponds to the quantity/concentration data of a corresponding analyte.

Preferably, said plurality of analytes of dataset DS are defined by a plurality of odorous analytes present in the packaged edible oil extracted from olives, preferably of a virgin or extra-virgin oil.

Preferably, the analytes of the DS dataset are analyzed by the solid phase micro-extraction (SPME) headspace technique.

Preferably, the DS dataset analyte amount/concentration data are obtained by the headspace technique (HS) with solid phase micro-extraction (SPME) coupled with a gas chromatograph (GC) and mass spectrometer (MS); said technique being also known by the acronym “HS-SPME-GC/MS”.

Preferably, the quantity/concentration data of the DS dataset analytes are obtained by the HS-SPME-GC/MS technique as described in “Multiple internal standard normalization for improving HS-SPME-GC-MS quantitation in virgin olive oil volatile organic compounds (VOO-VOCs) profile” by Martina Fortinia, Marzia Migliorini, Chiara Cherubini, Lorenzo Cecchi, Luca Calamai, Talanta Volume 165, 1 Apr. 2017, Pages 641-652.

Conveniently, the quantities/concentrations of said plurality of analytes of the DS dataset are obtained by the following procedure:
a specific quantity of each oil sample to be analyzed is inserted into a container, for example a phial (vial) with a suitable septum/closing cap, and preferably a predefined and suitable quantity of a reference standard can also be added to the inside of said container,
an SPME fiber is exposed—preferably after having thermostated the vessel containing the oil sample to be analyzed—in the headspace of the closed vessel for a predefined time (e.g. 20 minutes) and under stirring (e.g. at a stirring speed of 400-500 rpm),
the SPME fiber is desorbed for a predefined period (e.g. 2 sec-2 minutes) in the injection port of a gas chromatograph which, preferably, operates in “splitless” mode at an injection temperature of 260° C.,
the analytes are identified and quantified, in a traditional way, by means of gas chromatography coupled to mass spectrometry.

Preferably, the SPME fiber is DVB/CAR/PDMS (Divinylbenzene/Carboxen/Polydimethylsiloxane).

Preferably, the gas chromatograph column has a thickness of 0.25 mm, a length of 56 m and a diameter of 0.25 mm.

Preferably, the gas chromatograph is configured to operate in the following modes:
oven column temperature: 35° C.;
injection temperature: 260° C.;
injection mode: splitless;
sampling time: 0.05 min;
carrier gas: He;
split ratio: 50.

Preferably, the initial temperature of the GC column is maintained at 36° C. for 15 minutes, then increased to 160° C. with an increase of 4°/minute and a holding time of 1 minute, then increased to 300° C. with an increase of 50°/minute and a holding time of about 1 minute.

Preferably, mass spectrometry is performed using a quadrupole mass analyzer.

Preferably, in another possible embodiment, the odorous analytes of the DS dataset are analyzed by the headspace technique (HS) coupled with a gas chromatograph (GC) and an ion mobility spectrometer (IMS), also known as “HS-GC-IMS”.

Suitably, therefore, the spectrometer used for the analysis coupled with the gas chromatograph can be a mass spectrometer (MS) or an ion mobility spectrometer (IMS).

Preferably, the odorous analytes of the DS dataset (of which—for each—the corresponding quantity/concentration present in a sample of oil analyzed at different known and defined times with respect to the packaging date is acquired) comprise at least ten analytes, more preferably they comprise at least 50 analytes and, even more preferably, comprise 70-80 analytes.

Preferably, in one possible embodiment, the DS dataset analytes comprise: Acetic acid, methyl ester; 1-propanol; 2-Butanone; Acetic acid; 2-butanol; Ethyl acetate; 1-propanol, 2-methyl-; methyl propionate; Butanal, 3-methyl-; Butanal, 2-methyl-; 1-penten-3-ol; 1-penten-3-one (ethyl vinyl ketone); Propanoic acid; 3-Pentanone; Pentanal; heptane; (R)-(−)-2-pentanol; Propanoic acid, ethyl ester; 1-butanol, 3-methyl-; 1-butanol, 2-methyl-; 2-penalty, (E)-; 1-pentanol; 2-penten-1-ol, (E)-; 2-penten-1-ol, (Z)—; butanoic acid; Octane; exhale; Butanoic acid; ethyl ester; Acetic acid; butyl ester; 3-hexenal, (Z)—; 2-hexenal, (E)-; 3-hexen-1-ol, (E)-; 3-hexen-1-ol, (Z)—; 2-hexen-1-ol, (E)-; 2-hexen-1-ol, (Z)—; 1-hexanol; pentanoic acid; 2-heptanone; heptanal; 2-heptanol; 2;4-hexadienal, (E;E)-; 2-Heptenal, (E)-; Benzaldehyde; 1-heptanol; 1-Oct-3-one; Hexanoic acid; Phenol; 5-heptene-2-one, 6-methyl-; 1-octene-3-ol; 2-octanone; 2-octanol; octal; 3-hexen-1-ol; acetate; (Z)—; 2;4-heptadienal, (E;E)-; Acetic acid; thin ester; 2-hexen-1-ol; acetate, (E)-; D-Limonene; 2-octal, (E)-; 1-octanol; Phenol, 2-methoxy-(Guaiacol); 2-Nonanone; Not anal; Phenylethyl alcohol; 2-Nonenal; (AND)-; Phenol; 4-ethyl-; 1-Nonanol; Decalogue; 2;4-Nonadienal, (E;E)-; 2-Decenal, (E)-; Phenol, 4-ethyl-2-methoxy-; 2;4-Decadienal, (E;E)-.

Preferably, in another possible embodiment, the analytes of dataset DS also comprise, in addition to those reported above, the following further analytes: acetic acid-d3; trimethylacetaldehyde; Ethyl acetate-d8; butanol-d10; toluene-D8; 2-pentanol, 4-methyl; 3-octanone; 6-chloro-2-hexanone; Phenol, 3,4-dimethyl.

Preferably, the numerical value (label) of the DS dataset indicates the number of months which have elapsed between the packaging date and the moment in which the analysis was carried out.

Preferably, the numerical value (label) of the DS dataset can represent, or be connected to, the “minimum conservation term” (TMC) which—according to the relevant regulations (for example EU Reg. 1169/2011) must be reported on the label of food products and which is defined as “the date until which a product retains its specific properties under suitable storage conditions”. In particular, as far as extra virgin olive oil is concerned, these specific properties can be found in the legislation which defines the qualitative characteristics of an extra virgin olive oil, and more in detail in the following current regulations: EEC Regulation 2568/91 and Delegated Regulation (EU) 2015/1830).

Preferably, the dataset DS comprises data of a packaged oil of at least one type (more preferably of several types that are different from each other and obtained by mixing different oils, i.e. obtained from different varieties of olives) and taken from different/corresponding packages, to be thus analyzed in a series of moments following its packaging date.

In particular, the DS dataset data include the quantity/concentration values of a plurality of predefined analytes—preferably of the plurality of analytes as indicated above—which, for each oil sample, were analyzed at various known and predefined time intervals compared to the packaging/bottling date, where in particular the numerical value of each label corresponds to the corresponding number of months/days elapsed between the date on which the bottle/package was opened to carry out the analysis and the bottling/packaging date. For example, in data from a possible DS dataset:
the label with numerical value “0” corresponds to the same month of bottling,
the label with numerical value “4” corresponds to an analysis carried out 4 months after the month of bottling,
the label with numerical value “8” corresponds to an analysis carried out 8 months after the month of bottling,
the label with numerical value “12” corresponds to an analysis carried out 12 months after the month of bottling,
the label with numerical value “16” corresponds to an analysis carried out 16 months after the month of bottling,
the label with numerical value “20” corresponds to an analysis carried out 20 months after the month of bottling.

In more detail, for example, the DS dataset can include the analyte data of twelve different types of extra virgin olive oils, and in which each type of oil has been packaged/bottled in six bottles/packages. Each of the six bottles/packs of oil of a specific type was therefore opened at a different time compared to the remaining bottles/packs of oil of the same type, in order to thus be able to carry out a corresponding analysis using the headspace technique (HS) in coupling with a gas chromatograph and a spectrometer, preferably by means of the HS-SPME-GC/MS technique, in one of the aforementioned six known and successive moments. In more detail, for each of the twelve types of oil, a first bottle of oil of a certain type was opened and analyzed in the same month of bottling (“0”), a second bottle of oil of the same type was opened and analyzed 4 months after bottling (“4”), a third bottle of oil of the same type was opened and analyzed 8 months after bottling (“8”), a fourth bottle of oil of the same type was opened and analyzed 12 months after bottling (“12”), a fifth bottle of oil of the same type was opened and analyzed 16 months after bottling (“16”), a sixth bottle of oil of the same type was opened and analyzed 20 months after bottling (“20”). Conveniently, all the aforementioned bottles/packages of oil have been stored over time in the typical conditions of a warehouse (at a temperature of 16-20° C.) in closed cartons, each of which contains six bottles of oil of the same type.

Preferably, the DS dataset includes data relating to oil samples (S1) that have not been placed on the market (and which are then analyzed at subsequent moments but without being placed on the market, thus ensuring their conservation in known and predefined conditions) and also data relating to samples of off-the-shelf oils (S2), i.e. actually placed on the market, to thus introduce into the DS dataset the variability deriving from the conditions of transport and/or conservation and/or other factors. For example, the DS dataset includes approximately 80% data relating to samples of oils not placed on the market (S1) and approximately 20% data relating to samples of oils on the shelf (S2).

Advantageously, the DS dataset can be elaborated/processed with the SMOTE algorithm (“Synthetic Minority Oversampling”) so that it is suitably balanced.

Advantageously, the DS dataset can be further processed/processed in order to standardize and normalize the data.

Conveniently, each analysis of an oil sample of the DS dataset is defined by a corresponding record and, therefore, the DS dataset has a number of R records, where each record is defined by:
the quantity/concentration values of each analyte (features) of said plurality of predefined analytes which are analyzed according to the headspace technique (HS) coupled with a gas chromatograph and a spectrometer, preferably using the H S-SPME-GC technique/MS,
a numerical label (label) which thus defines the corresponding time elapsed between the packaging date and the date on which the analysis was carried out which led to the determination of the corresponding quantities/concentration of each analyte.

Conveniently, the ML model is trained using a training set TrS which is obtained from the dataset DS, in particular it is defined by a subset of said dataset DS. Preferably, the DS dataset is subdivided randomly into training set TrS and test set TeS and in such a way that the training set TrS has a much greater number of data/events than the test set TeS, for example it is subdivided so that approximately 70% of the total DS dataset is training set TrS and about 30% is test set TeS.

Conveniently, the characteristics—which as mentioned correspond to the quantity/concentration values of each analyte of said plurality of analytes which are analyzed according to the headspace technique (HS) coupled with a gas chromatograph and a spectrometer, preferably by the H S-SPME-GC/MS technique—are used as input variables of the training set TrS used to train at least one ML machine learning model which, preferably, uses the adaptive amplification regression algorithm (also called “adaboost regression”) and/or random forest regression (also called “random forest regression”). Advantageously, a first ML1 machine learning model that uses the adaptive amplification regression algorithm (also called “adaboost regression”) can be trained and then used and also, to further validate the result, a second model can be trained and used of ML2 machine learning that uses the random forest regression algorithm (also called “random forest regression”).

The DS dataset used—and advantageously the fact of using a plurality of analytes as features of the DS dataset, and in particular about 70-80 analytes, without having made their prior selection, as well as the fact of having built the DS dataset both with samples not placed on the market (and thus stored in known and identical conditions) and with real samples placed on the market (and stored in unknown and different conditions)—it has made it possible to train the ML machine learning model in balanced way and, at the same time, the thus trained ML machine learning model is accurate in terms of its ability to determine the elapsed time since the oil was packaged.

Conveniently, it is understood that the same type of extraction/analysis that is carried out, during the training phase, on the oil samples in order to obtain the quantity/concentration values of the corresponding analytes which thus define the characteristics F of the dataset DS is also performed, during the inference phase, on the oil sample X whose time elapsed since packaging is to be determined using the machine ML model trained using said training set TrS which is obtained by dividing the dataset DS.

Preferably, the same extractions and analyzes of the analytes in order to determine their corresponding quantities/concentrations, as well as possibly the same processing, which are carried out in order to obtain the characteristics (features) F of the DS dataset are also carried out, during the phase of inference, on the oil sample X whose time elapsed since packaging is to be determined using the machine ML model trained using said training set TrS which is obtained by dividing the dataset DS, to thus define the inputs I for the model inference phase ML machine learning, preferably an ML machine learning model using the adaptive amplification regression algorithm (also called “adaboost regression”) and/or random forest regression (also called “random forest regression”).

In more detail, the inputs I are defined by a set of data relating to the quantities/concentrations of the analytes which are obtained by carrying out, on the oil sample X whose time elapsed since packaging has to be determined, the same analyzes and processing which are carried out for each analyzed and labeled oil sample from the DS dataset.

Basically, the inputs I used in the inference phase of the ML machine learning model, and this in order to determine the time elapsed since the packaging of a given sample of packaged oil, have a format corresponding to the characteristics (features) F of the DS dataset, and in particular of the training set TrS used for the training phase of the model itself. In other words, the same analysis technique and also the same analytes that are used to obtain the characteristics (features) F of the DS dataset, and in particular of the training set TrS to be used for training the ML machine learning model, are also carried out to obtain the inputs I to be sent as input to the thus trained ML model in order to determine the time elapsed since packaging for a given sample of packaged oil X.

Preferably, the ML model which is trained with the TrS training set (which is obtained by dividing the DS dataset into said TrS training set and the TeS test set), is then tested by the TeS test set to determine the accuracy and effectiveness indices. Conveniently, for example, the coefficient of determination (R2) was 0.971 for training and 0.858 for the test, while the mean absolute error (MAE) was 0.582 for training and 1.268 for the test.

Preferably, as mentioned, the ML machine learning model uses the adaptive amplification regression algorithm (also called “adaboost regression”) and/or the random forest regression algorithm (also called “random forest regression”).

More preferably, for example, the “adaboost regression” algorithm can be characterized:
using 10,000 as a condition of the number of appraisers,
using as optimization criterion the loss function (also called “loss function”) of linear type.

More preferably, for example, the “random forest regression” algorithm can be characterized:
using 90,000 as a condition of the number of appraisers,
using as optimization criterion the absolute error function (also called “absolute error”).

Conveniently, as mentioned, the dataset DS is subdivided randomly into training set TrS and test set TeS and in such a way that the training set TrS has a greater number of data than the test set TeS, for example it is subdivided so that approximately 70% of the total DS dataset is training set TrS and about 30% is test set TeS.

Preferably, with the training set TrS the training of the ML model is carried out, as described above, as well as the tuning of the hyperparameters by cross-validation is also carried out.

Conveniently, the values of the hyperparameters of the ML machine learning model are then selected that provide the best score in the inference phase, in particular in terms of the coefficient of determination (also called “R2”) and for training (also called “training”), and for the verification phase (also called “testing”), as well as the robust index of the mean absolute error (also called “mean absolute error”).

Conveniently, the training and tuning phase is carried out in order to select the parameters which, in the training phase, minimize the mean absolute error and maximize the coefficient of determination.

Conveniently, the data from the TeS test set are instead then used to evaluate the performance of the ML model that has been trained.

Advantageously, the ML model was finally validated using the concentration/quantity data of the same analytes—extracted and analyzed in the same way as for the TrS training set—of packaged oils that have been placed on the market and, for example, that are available on shelf in the United States of America.

Advantageously, the trained and validated ML model can be uploaded to the cloud or to a shared remote drive which is accessible from various local terminals.

Conveniently, in the presence of two machine learning models ML1 and ML2 that use different algorithms, as preferably described above, the inference and/or test and/or validation phase is performed for each of the models, thus obtaining a corresponding value numerical output Tx1 and Tx2 for each of said models, and then select the numerical value which minimizes the average absolute error and maximizes the coefficient of determination which are calculated, for each model, during the respective inference and/or test phase and/or validation.

Alternatively, the ML machine learning model can use other supervised learning algorithms, such as:
neural network, for example “deep neural networks” (DNN).
support vector machines (SVM),
“k-nearest neighbors”,
“Quadratic Discriminant Analysis”,
Naive Bayes Gaussian.
“Ridge Regression”.

Preferably, according to the present invention, said at least one machine learning model ML (or ML1 and ML2 jointly) uses the adaptive amplification regression algorithm (also called “adaboost regression”) and/or random forest regression (also called “random forest regression”), as these are the optimal solution in terms of prediction accuracy (in particular in terms of the coefficient of determination in the test/verification phase) and stability in prediction. Furthermore, the solution that involves the joint use of two models ML1 and ML2, which respectively use the adaptive amplification regression algorithm (also called “adaboost regression”) and/or random forest regression (also called “random forest regression”), is also optimal both in terms of simplicity of implementation at the level of software instructions and of memory space required within the processor during model training, as well as of the subsequent distribution phase (also called “deployment”) for the subsequent use.

More in detail, after training the ML machine learning models with various algorithms using the same TrS training set, the following performances were obtained for the inference phase of each algorithm performed using the same TeS test set as input for all:

| machine learning | |
|---|---|
| algorithm | Performance indices |
| Decision tree | R² train: 1.000; R² test: 0.573 |
| | MAE train: 0.0001; MAE test 2.545 |
| Support vector | R² train: 0.145; R² test: 0.099 |
| machines (SVM) | MAE train: 5.503; MAE test 5.644 |
| Random forest | R² train: 0.985; R² test: 0.896 |
| | MAE train: 0.684; MAE test 1.553 |
| K-nearest | R² train: 0.573; R² test: 0.575 |
| neighbors | MAE train: 3.827; MAE test 3.273 |
| regression | |
| Adaboost | R² train: 0.991; R² test: 0.943 |
| regression | MAE train: 0.342; MAE test 0.922 |
| Quadratic | Collinearity highlighted |
| Discriminant | |
| Analysis | |
| Naive Bayes | R² train: 0.972; R² test: 0.954 |
| Gaussian | MAE train: 0.320; MAE test 0.545 |
| Ridge regression | R² train: 0.849; R² test: 0.622 |
| | MAE train: 2.609; MAE test 3.128 |
| Neural network of | Very low accuracy and loss: 10.585 |
| the DNN type with | |
| 7 hidden layers | |

This confirms that the adaptive amplification regression algorithm and/or the random forest regression algorithm turn out to be the optimal solution as they present the values of the coefficient of determination (also called “R²”) in training, but above all in verification or testing, the highest ever, respectively 0.943 and 0.896, thus ensuring an excellent prediction estimated through the evaluation of the parameters of the linear interpolation line between the values predicted by the algorithm and the real values of months of life of a data set of validation completely different from the training and verification sets used for the determination of the optimal parameters of the algorithms.

In particular, the random forest algorithm is very effective in terms of predictability and stability for the entire time span used, while the adaptive amplification algorithm (also called “adaboost”) is extremely effective especially for the initial and final values of the analyzed lifetime. Therefore, advantageously, by operating jointly, the two algorithms allow the validation of the result in a more effective and secure manner since the selection of the numerical value Tx1 or Tx2 is always carried out considering the one with the best performance indexes.

Unlike traditional methods, the method according to the invention considers the quantities/concentrations of a plurality of analytes (at least 70-80 analytes, or even more) in an “untargeted” way, thus avoiding building the prediction model by selecting and choosing a specific analyte or a subset of analytes. Conveniently, in the method according to the invention no “feature selection” is envisaged among the plurality of analytes extracted and analyzed by means of the headspace technique coupled with a gas chromatograph and a spectrometer and, preferably, by means of the H S-SPME technique -GC/MS. In other words, the concentrations of all odorous analytes, and preferably of about 70-80 predefined analytes (or even more), of each oil sample analyzed by the headspace technique coupled with a gas chromatograph and spectrometer are then use all of them as they are both as features of the DS dataset in the training phase and as input I in the inference phase.

From what has been said, the method according to the invention is particularly advantageous in that:
it is simple, effective and based on easily determined data with good repeatability,
is accurate and stable,
has a low cost of application,
the use of a DS training dataset including data of packaged oils placed on the market allows the ML model to be kept anchored to the distribution reality and to the factors that could intervene, after packaging, in the transport and storage phase (for example road transport, ship, shelf arrival, etc.)
it can be made available to the entire industry, particularly through cloud computing.

Suitably, the present invention also relates to a processing unit device in which a software module configured to implement a method for determining the time which has elapsed since the packaging of an edible oil which is packaged and which is extracted from olives, preferably of a virgin or extra-virgin packaged oil, as described above in its essential and/or preferential aspects.

The present invention also relates to a computer program comprising instructions which, when the program is executed on a computer, causes the computer itself to execute a method for determining the time which has elapsed since the packaging of a packaged edible oil extracted from olives, preferably of a packaged virgin or extra-virgin oil, as described above in its essential and/or preferential aspects.

The present invention also relates to a computer-readable medium comprising instructions which, when the program is executed on a computer, causes the computer itself to execute a method for determining the time which has elapsed since the packaging of an edible oil packaged and extracted from olives, preferably of a packaged virgin or extra-virgin oil, as described above in its essential and/or preferential aspects.

The present invention also relates to a method for training an ML machine learning model to determine the time which has elapsed since the packaging of a packaged edible oil extracted from olives, preferably of a packaged virgin or extra-virgin oil, in which said method is trained using a TrS training set which is obtained from a DS dataset, as described above in its essential and/or preferred aspects.

Conveniently, in a possible embodiment, the processing unit of a device is configured to implement a training method of an ML machine learning model as described above in its essential and/or preferential aspects.

The present invention also relates to a computer program comprising instructions which, when the program is executed on a computer, causes the computer itself to execute the training method of an ML machine learning model described above in its essential and/or preferential aspects.

The present invention also relates to a computer-readable medium comprising instructions which, when the program is executed on a computer, lead the computer itself to execute the training method of an ML machine learning model described above in its essential aspects and/or preferential.

Conveniently, in a possible embodiment, a software module is loaded into the processing unit of the device with the ML machine learning model which has already been trained with the training method described above in its essential and/or preferential aspects.

Conveniently, the present invention also relates to a method, implemented by at least one computer, for determining the time which has elapsed since the packaging of a packaged edible oil extracted from olives, preferably of a packaged virgin or extra-virgin oil, said method envisaging the use an ML machine learning model, which has been previously trained using a TrS training set which is obtained from a DS dataset as described above in its essential and/or preferential aspects, and in which said method provides for:
supply as input I to said ML machine learning model, which has been previously trained, the quantity/concentration data of a plurality of predefined analytes contained in a sample of oil X and which are obtained by carrying out an analysis according to the space technique head coupled with a gas chromatograph and a spectrometer,
receive in output O from said machine learning model ML an indication, in particular in the form of a label in numerical format, preferably defined by an integer, which is representative and/or corresponds to the time which has elapsed between the packaging of said sample of oil X and the moment in which said analysis was carried out.

The present invention has been illustrated and described in some of its preferred embodiments, but it is understood that executive variants can be applied to them in practice, without however departing from the scope of protection of the present patent for industrial invention.

1. Method, implemented by at least one computer, for determining the time which has elapsed since the packaging of an edible oil (X) which is packaged and which is extracted from olives, said method comprising:
training at least one machine learning (ML) model to determine the time that has elapsed since the packaging of a packaged edible oil extracted from olives,
analyzing a sample of said oil (X), for which it is desired to determine the time elapsed since its packaging, to acquire the quantity/concentration data of a plurality of analytes contained in said oil sample (X), said oil sample (X) being analyzed by a headspace technique coupled with a gas chromatograph and spectrometer,
sending the data acquired in this way, and relating to the quantities/concentrations of said plurality of analytes in said sample of oil (X), in input (I), to said at least one machine learning model (ML), already trained, to thus obtain at an output (O) a numerical value (Tx) corresponding to, or representative of, the time which has elapsed since the packaging of said oil (X).

2. The method according to claim 1, that further comprising:
training a first machine learning model (ML1) with an adaptive amplification regression algorithm,
training a second machine learning model (ML2) with an algorithm that is different from the first machine learning model (ML1), preferably train a second machine learning model (ML2) with a random forest regression algorithm,
sending the data acquired in this way, and relating to the quantities/concentrations of said plurality of analytes in the oil sample (X), in input (I), to said first machine learning model (ML1) to thus obtain at an output (O1) of said first machine learning module (ML1), a first numerical value (Tx1) corresponding to, or representative of, the time that has elapsed since the packaging of said oil sample (X),
sending the data acquired in this way, and relating to the quantities/concentrations of said plurality of analytes in the oil sample (X), in the input (I), to said second machine learning model (ML2) to thus obtain at an output (O2) of said second machine learning module (ML2), a second numerical value (Tx2) corresponding to, or representative of, the time that has elapsed since the packaging of said oil sample (X),
selecting, if the first numerical value (Tx1) is different from the second numerical value (Tx2), one of said first numerical value (Tx1) or said second numerical value (Tx2) on the basis of performance indexes of the respective machine learning models (ML1, ML2).

3. The method according to claim 2, wherein said first machine learning model (ML1) is trained with an algorithm of the adaptive amplification regression type.

4. The method according to claim 2, wherein said second machine learning model (ML2) is trained with a random forest regression type algorithm.

5. The method according to claim 1, wherein said at least one machine learning model (ML, ML1, ML2) is trained using a dataset DS for supervised learning, and using a training set TrS which is defined by a subset of said DS dataset, comprising quantity/concentration data of said plurality of analytes and relating to various oil samples, of the same typology and preferably also of different typologies, which are analyzed, by means of the headspace technique in coupling with a gas chromatograph and a spectrometer, at a known moment which is defined starting from the packaging date of each oil sample.

6. The method according to claim 1, wherein said at least one machine learning model (ML, ML1, ML2) is trained using a dataset DS comprising a plurality of records (R), one for each analysis performed on a sample of oil, and each record includes:
as characteristics the quantity/concentration data relating to each analyte of said plurality of analytes analyzed by means of the headspace coupled with a gas chromatograph and a spectrometer,
a numerical value and expressed in months and/or days, which indicates the time elapsed between the packaging date and the date/moment in which the analysis was carried out which led to the determination of the corresponding amount/concentration of analytes.

7. The method according to claim 1, wherein said dataset DS for training said at least one machine learning model (ML, ML1, ML2) comprises:
data relating to oil samples (S1) that have not been placed on the market, thus resulting stored in known and predefined conditions, and
data relating to oil samples (S2) that have been placed on the market, in order to introduce in the DS dataset, in this way to introduce in the DS dataset the variability deriving from the transport and/or storage conditions and/or other factors.

8. The method according to claim 1, wherein said analytes of said oil sample (X) and of the oil samples of the training dataset DS are analyzed by means of the headspace technique with phase micro-extraction solid (SPME).

9. The method according to claim 1, wherein said spectrometer is a mass spectrometer (MS) or an ion mobility spectrometer (IMS).

10. The method according to claim 1, wherein said numerical value (Tx, Tx1, Tx2) output from said at least one machine learning model (ML, ML1, ML2) is processed so as to determine the time residual (Tm) with respect to a predefined maximum duration time (Tmax).

11. The method according to claim 1, wherein said numerical value (Tx, Tx1, Tx2) output from said at least one machine learning model (ML, ML1, ML2) is representative and/or is used to calculate or evaluate the shelf life (TMC) of said edible oil (X).

12. The method according to claim 1, wherein said plurality of analytes of said oil sample (X) and of the oil samples of the training dataset DS, and of which the quantity/concentration data are acquired, include at least 10 of the following analytes: Acetic acid, methyl ester; 1-propanol; 2-Butanone; Acetic acid; 2-butanol; Ethyl acetate; 1-propanol, 2-methyl-; methyl propionate; Butanal, 3-methyl-; Butanal, 2-methyl-; 1-penten-3-ol; 1-penten-3-one (ethyl vinyl ketone); Propanoic acid; 3-Pentanone; Pentanal; heptane; (R)-(−)-2-pentanol; Propanoic acid, ethyl ester; 1-butanol, 3-methyl-; 1-butanol, 2-methyl-; 2-penalty, (E)-; 1-pentanol; 2-penten-1-ol, (E)-; 2-penten-1-ol, (Z)—; butanoic acid; Octane; exhale; Butanoic acid; ethyl ester; Acetic acid; butyl ester; 3-hexenal, (Z)—; 2-hexenal, (E)-; 3-hexen-1-ol, (E)-; 3-hexen-1-ol, (Z)—; 2-hexen-1-ol, (E)-; 2-hexen-1-ol, (Z)—; 1-hexanol; pentanoic acid; 2-heptanone; heptanal; 2-heptanol; 2;4-hexadienal, (E;E)-; 2-Heptenal, (E)-; Benzaldehyde; 1-heptanol; 1-Oct-3-one; Hexanoic acid; Phenol; 5-heptene-2-one, 6-methyl-; 1-octene-3-ol; 2-octanone; 2-octanol; octal; 3-hexen-1-ol; acetate; (Z)—; 2;4-heptadienal, (E;E)-; Acetic acid; thin ester; 2-hexen-1-ol; acetate, (E)-; D-Limonene; 2-octal, (E)-; 1-octanol; Phenol, 2-methoxy-(Guaiacol); 2-Nonanone; Not anal; Phenylethyl alcohol; 2-Nonenal; (AND)-; Phenol; 4-ethyl-; 1-Nonanol; Decalogue; 2;4-Nonadienal, (E;E)-; 2-Decenal, (E)-; Phenol, 4-ethyl-2-methoxy-; 2;4-Decadienal, (E;E)-.

13. The method according to claim 1, wherein said plurality of analytes of said oil sample (X) and of the oil samples of the training dataset DS, and of which the quantity/concentration data are acquired, include at least 50 of the following analytes: Acetic acid, methyl ester; 1-propanol; 2-Butanone; Acetic acid; 2-butanol; Ethyl acetate; 1-propanol, 2-methyl-; methyl propionate; Butanal, 3-methyl-; Butanal, 2-methyl-; 1-penten-3-ol; 1-penten-3-one (ethyl vinyl ketone); Propanoic acid; 3-Pentanone; Pentanal; heptane; (R)-(−)-2-pentanol; Propanoic acid, ethyl ester; 1-butanol, 3-methyl-; 1-butanol, 2-methyl-; 2-penalty, (E)-; 1-pentanol; 2-penten-1-ol, (E)-; 2-penten-1-ol, (Z)—; butanoic acid; Octane; exhale; Butanoic acid; ethyl ester; Acetic acid; butyl ester; 3-hexenal, (Z)—; 2-hexenal, (E)-; 3-hexen-1-ol, (E)-; 3-hexen-1-ol, (Z)—; 2-hexen-1-ol, (E)-; 2-hexen-1-ol, (Z)—; 1-hexanol; pentanoic acid; 2-heptanone; heptanal; 2-heptanol; 2;4-hexadienal, (E;E)-; 2-Heptenal, (E)-; Benzaldehyde; 1-heptanol; 1-Oct-3-one; Hexanoic acid; Phenol; 5-heptene-2-one, 6-methyl-; 1-octene-3-ol; 2-octanone; 2-octanol; octal; 3-hexen-1-ol; acetate; (Z)—; 2;4-heptadienal, (E;E)-; Acetic acid; thin ester; 2-hexen-1-ol; acetate, (E)-; D-Limonene; 2-octal, (E)-; 1-octanol; Phenol, 2-methoxy-(Guaiacol); 2-Nonanone; Not anal; Phenylethyl alcohol; 2-Nonenal; (AND)-; Phenol; 4-ethyl-; 1-Nonanol; Decalogue; 2;4-Nonadienal, (E;E)-; 2-Decenal, (E)-; Phenol, 4-ethyl-2-methoxy-; 2;4-Decadienal, (E;E)-.

14. The method according to claim 1, wherein said plurality of analytes of said oil sample (X) and of the oil samples of the training dataset DS, and of which the quantity/concentration data are acquired, include all of the following analytes: Acetic acid, methyl ester; 1-propanol; 2-Butanone; Acetic acid; 2-butanol; Ethyl acetate; 1-propanol, 2-methyl-; methyl propionate; Butanal, 3-methyl-; Butanal, 2-methyl-; 1-penten-3-ol; 1-penten-3-one (ethyl vinyl ketone); Propanoic acid; 3-Pentanone; Pentanal; heptane; (R)-(−)-2-pentanol; Propanoic acid, ethyl ester; 1-butanol, 3-methyl-; 1-butanol, 2-methyl-; 2-penalty, (E)-; 1-pentanol; 2-penten-1-ol, (E)-; 2-penten-1-ol, (Z)—; butanoic acid; Octane; exhale; Butanoic acid; ethyl ester; Acetic acid; butyl ester; 3-hexenal, (Z)—; 2-hexenal, (E)-; 3-hexen-1-ol, (E)-; 3-hexen-1-ol, (Z)—; 2-hexen-1-ol, (E)-; 2-hexen-1-ol, (Z)—; 1-hexanol; pentanoic acid; 2-heptanone; heptanal; 2-heptanol; 2;4-hexadienal, (E;E)-; 2-Heptenal, (E)-; Benzaldehyde; 1-heptanol; 1-Oct-3-one; Hexanoic acid; Phenol; 5-heptene-2-one, 6-methyl-; 1-octene-3-ol; 2-octanone; 2-octanol; octal; 3-hexen-1-ol; acetate; (Z)—; 2;4-heptadienal, (E;E)-; Acetic acid; thin ester; 2-hexen-1-ol; acetate, (E)-; D-Limonene; 2-octal, (E)-; 1-octanol; Phenol, 2-methoxy-(Guaiacol); 2-Nonanone; Not anal; Phenylethyl alcohol; 2-Nonenal; (AND)-; Phenol; 4-ethyl-; 1-Nonanol; Decalogue; 2;4-Nonadienal, (E;E)-; 2-Decenal, (E)-; Phenol, 4-ethyl-2-methoxy-; 2;4-Decadienal, (E;E)-.

15. The method according to claim 1, wherein the quantities/concentrations of said plurality of analytes present in a sample of oil X are obtained by the following:
placing a certain quantity of the oil sample X to be analyzed in a container,
exposing a SPME fiber in the headspace of the closed vessel for a predefined time and under stirring,
desorbing the SPME fiber for a predefined period in an injection port of a gas chromatograph,
identifying quantities/concentrations of the analytes and determined by gas chromatography coupled to mass spectrometry.
